(19) United States
(12) Patent Application Publication
(10) Pub. No.: US 2017/0107260 A1
(43) Pub. Date: Apr. 20, 2017

(54) MOSAIC HIV-1 SEQUENCES AND USES THEREOF

(71) Applicants: Duke University, Durham, NC (US); Los Alamos National Security, LLC, Los Alamos, NM (US)

(72) Inventors: Barton F. Haynes, Durham, NC (US); Bette T. Korber, Los Alamos, NM (US)

(21) Appl. No.: 15/127,986

(22) PCT Filed: Mar. 25, 2015
(86) PCT No.: PCT/US2015/022380

§ 371 Date: Sep. 21, 2016

Related U.S. Application Data
(60) Provisional application No. 61/969,954, filed on Mar. 25, 2014.

(51) Int. Cl. C07K 14/005; A61K 39/21

(52) U.S. Cl. CPC C07K 14/005; A61K 39/21; C12N 2740/16034; A61K 2039/70

(57) ABSTRACT

The invention is directed to mosaic HIV 1 genes designed to induce T-cell responses.

This application claims the benefit of priority of U.S. Application Ser. No. 61/969,954 filed Mar. 25, 2014, the contents of which application is herein incorporated by reference in its entirety.

This invention was made with government support under Center for HIV/AIDS Vaccine Immunology-Immunogen Design grant UM1-AI100645 from the NIH, NIAID, Division of AIDS. The government has certain rights in the invention.

### FIELD OF THE INVENTION

The invention provides compositions, methods, and kits for the treatment or prevention of viral infections. The polyvalent (e.g., 2-valent) immunogenic compositions and methods described herein incorporate computationally-optimized HIV-1 polypeptides and nucleic acid sequences encoding these that can increase the diversity, breadth and depth of cellular immune response in vaccinated subjects.

### BACKGROUND

Vaccines that elicit cellular immune responses against viruses must reflect global viral diversity in order to effectively treat or prevent viral infection. For example, the initiation of intense and diverse HIV-1-specific T cell responses is likely crucial for an effective HIV-1 vaccine. Cytotoxic T lymphocyte (CTL) responses are correlated with slow disease progression in humans, and the importance of CTL responses in non-human primate vaccination models is well established. While the highly variable Envelope (Env) is the primary target for neutralizing antibodies against HIV and vaccine antigens will also need to be tailored to elicit these antibody responses. T cell vaccine components can target more conserved proteins to trigger responses that are more likely to cross-react. But even the most conserved HIV-1 proteins are diverse enough that variation will be an issue.

Designing an effective HIV vaccine is a many-faceted challenge. The vaccine preferably elicits an immune response capable of either preventing infection or, minimally, controlling viral replication if infection occurs, despite the failure of immune responses to natural infection to eliminate the virus (Nabel, Vaccine 20:1945-1947 (2002)) or to protect from superinfection (Altfeld et al, Nature 420:434-439 (2002)). Potent vaccines are needed, with optimized vectors, immunization protocols, and adjuvants (Nabel, Vaccine 20:1945-1947 (2002)), combined with antigens that can stimulate cross-reactive responses against the diverse spectrum of circulating viruses (Gaschen et al, Science 296:2354-2360 (2002), Korber et al, Br. Med. Bull. 58:19-42 (2001)). The problems that influenza vaccinologists have confronted for decades highlight the challenge posed by HIV-1:human influenza strains undergoing antigenic drift diverge from one another by around 1-2% per year, yet vaccine antigens often fail to elicit cross-reactive B-cell responses from one year to the next, requiring that contemporary strains be continuously monitored and vaccines be updated every few years (Korber et al, Br. Med. Bull. 58:19-42 (2001)). In contrast, co-circulating individual HIV-1 strains can differ from one another by 20% or more in relatively conserved proteins, and up to 35% in the Envelope protein (Gaschen et al, Science 296:2354-2360 (2002). Korber et al. Br. Med. Bull. 58:19-42 (2001)).

Different degrees of viral diversity in regional HIV-1 epidemics provide a potentially useful hierarchy for vaccine design strategies. Some geographic regions recapitulate global diversity, with a majority of known HIV-1 subtypes, or clades, co-circulating (e g., the Democratic Republic of the Congo (Mokili & Korber, J. Neurovirol 11 (Suppl. 1):66-75 (2005)); others are dominated by two subtypes and their recombinants (e.g., Uganda (Barugahare et al, J. Virol. 79:4132-4139 (2005)), and others by a single subtype (e.g., South Africa (Williamson et al, AIDS Res. Hum. Retroviruses 19:133-144 (2003)). Even areas with predominantly single-subtype epidemics must address extensive within-clade diversity (Williamson et al, AIDS Res. Hum. Retroviruses 19:133-44 (2003)) but, since international travel can be expected to further blur geographic distinctions, all nations would benefit from a global vaccine.

### SUMMARY OF THE INVENTION

In certain aspects, the invention provides a composition comprising a nucleic acid encoding any one of the mosaic polypeptides of FIG. 1, or a combination thereof. In certain embodiments, the nucleic acids encoding the polypeptides of the invention are included as components to induce T-cell responses in an immunogenic composition which further comprises immunogen which induce humoral responses to HIV-1.

In certain aspects, the invention provides a composition comprising a bivalent set of nucleic acids encoding two mosaic polypeptides of FIG. 1, or any combination thereof, wherein the set of two mosaic polypeptides correspond to the same viral gene product. A composition comprising a bivalent set of nucleic acids encoding two mosaic polypeptides of FIG. 1, or any combination thereof, wherein the set of two mosaic polypeptides are for the same viral gene product.

In certain aspects the composition comprises nucleic acids encoding Gag.Mos.2.1, Gag.Mos.2.2, or the combination thereof. In certain aspects the composition comprises nucleic acids encoding Env.Mos.2.1, Env.Mos.2.2, or the combination thereof. In certain aspects the composition comprises nucleic acids encoding Vif.Mos.2.1, Vif.Mos.2.2, or the combination thereof. In certain aspects the composition comprises nucleic acids encoding Pol.Mos.2.1, Pol.Mos.2.2, or the combination thereof. In certain aspects the composition comprises nucleic acids encoding Nef.Mos.2.1, Nef.Mos.2.2, or the combination thereof. In certain aspects the composition comprises nucleic acids encoding Gag.Mos.2.1, Gag.Mos.2.2, Vif.Mos.2.1, Vif.Mos.2.2, or the combination thereof.

The composition of the invention can further comprise Nef.Mos.2.1, Nef.Mos.2.2, or the combination thereof. The composition of the invention can further comprise Env.Mos.2.1, Env.Mos.2.2, or the combination thereof. The composition of the invention can further comprise Gag.Mos.2.1, Gag.Mos.2.2, or the combination thereof. The composition of the invention can further comprise Vif.Mos.2.1, Vif.Mos.2.2, or the combination thereof.

The compositions of the invention can further comprise an adjuvant.

In certain embodiments, the composition further comprises an immunogen, for example but not limited to HIV1 envelope, suitable to elicit a humoral response.

In certain aspects, the invention provides a method of inducing an immune response in a subject comprising administering to the subject an amount of the composition of any one of claim 1-12 in an amount sufficient to effect such induction. In certain embodiments, the methods of the invention further comprising administering an immunogen, for example but not limited to HIV1 envelope, suitable to elicit a humoral response. In certain embodiments the immunogen is a transmitter founder HIV1 envelope. In certain embodiments the envelope is a CH505 envelope or a combination thereof as described in U.S. Ser. No. 61/955,402. The contents of this application is hereby incorporated by reference in its entirety. In certain embodiments the humoral response is HIV1 antibodies, wherein the antibodies neutralize the virus.

In certain embodiments, the compositions and methods of the invention comprise polypeptides instead of nucleic acids. In certain embodiments, the compositions and methods of the invention comprise a combination of nucleic acids and polypeptides.

### BRIEF DESCRIPTION OF THE DRAWINGS

FIG. 1 shows the M group 2 mosaic pairs (SEQ ID NOs: 1-18 in order of appearance) for every HIV of proteins based on the September 2013 Los Alamos database alignments, using T cell mosaic design tools (See U.S. Pat. No. 7,951,377).

### DETAILED DESCRIPTION

Presented herein is the design of polyvalent vaccine antigen sets focusing on T lymphocyte responses, optimized for either the common B and C subtypes, or all HIV-1 variants in global circulation [the HIV-1 Main (M) group]. Cytotoxic T-lymphocytes (CTL) directly kill infected, virus-producing host cells, recognizing them via viral protein fragments (epitopes) presented on infected cell surfaces by human leukocyte antigen (HLA) molecules. Helper T-cell responses control varied aspects of the immune response through the release of cytokines. Both are likely to be crucial for an HIV-1 vaccine: CTI, responses have been implicated in slowing disease progression (Oxenius et al, J. Infect. Dis. 189:1199-208 (2004)); vaccine-elicited cellular immune responses in nonhuman primates help control pathogenic SIV or SHIV, reducing the likelihood of disease after challenge (Barouch et al, Science 290:486-92 (2000)); and experimental depletion of CD8+ T-cells results in increased viremia in SIV infected rhesus macaques Schmitz et al, Science 283:857-60 (1999)). Furthermore, CTL escape mutations are associated with disease progression (Barouch et al, J. Virol, 77:7367-75 (2003)), thus vaccine-stimulated memory responses that block potential escape routes may be valuable.

The highly variable Env protein is the primary target for neutralizing antibodies against HIV: since immune protection will likely require both B-cell and T-cell responses (Moore and Burton, Nat. Med. 10:769-71 (2004)), Env vaccine antigens will also need to be optimized separately to elicit antibody responses. T-cell-directed vaccine components, in contrast, can target the more conserved proteins, but even the most conserved HIV-1 proteins are diverse enough that variation is an issue. Artificial central-sequence vaccine approaches (e.g., consensus sequences, in which every amino acid is found in a plurality of sequences, or maximum likelihood reconstructions of ancestral sequences (Gaschen et al, Science 296:2354-60 (2002), Gao et al, J. Virol. 79:1154-63 (2005), Doria-Rose et al, J. Virol. 79:11214-24 (2005). Weaver et at, J. Virol., in press)) are promising: nevertheless, even centralized strains provide limited coverage of HIV-1 variants, and consensus-based reagents fail to detect many autologous T-cell responses (Altfeld et al, J. Virol. 77:7330-40 (2003)).

Single amino acid changes can allow an epitope to escape T-cell surveillance; since many T-cell epitopes differ between HIV-1 strains at one or more positions, potential responses to any single vaccine antigen are limited. Whether a particular mutation results in escape depends upon the specific epitope/T-cell combination, although some changes broadly affect between-subtype cross-reactivity (Norris et al, AIDS Res. Hum. Retroviruses 20:315-25 (2004)). Including multiple variants in a polyvalent vaccine could enable responses to a broader range of circulating variants, and could also prime the immune system against common escape mutants (Jones et al, J. Exp. Med. 200:1243-56 (2004)). Escape from one T-cell receptor may create a variant that is susceptible to another (Allen et al, J. Virol. 79:12952-60 (2005), Feeney et al, J. Immunol. 174:7524-30 (2005)), so stimulating polyclonal responses to epitope variants may be beneficial (Killian et al, Aids 19:887-96 (2005)). Escape mutations that inhibit processing (Milicic et al, J. Immunol. 175:4618-26 (2005)) or HLA binding (Ammaranond et al, AIDS Res. Hum. Retroviruses 21:395-7 (2005)) cannot be directly countered by a T-cell with a different specificity, but responses to overlapping epitopes may block even some of these escape routes.

The present invention relates to a polyvalent vaccine comprising “mosaic” (optimized) proteins (or genes encoding these proteins). In certain embodiments, the vaccine composition comprises any one of the HIV1 genes, or any combination thereof. The candidate vaccine antigens can be cocktails of k composite proteins (k being the number of sequence variants in the cocktail), optimized to include the maximum number of potential T-cell epitopes in an input set of viral proteins. In certain embodiments k=2. The mosaics are designed from natural sequences: they resemble natural proteins and include the most common forms of potential epitopes. Since CD8+ epitopes are contiguous and typically nine amino-acids long, sets of mosaics can be scored by “coverage” of nonamers (9-mers) in the natural sequences (fragments of similar lengths are also well represented). 9-Mers not found at least three times can be excluded. This strategy of designing mosaics (optimized polypeptides) provides the level of diversity coverage achieved by a massively polyvalent multiple-peptide vaccine but with important advantages: it allows vaccine delivery as intact proteins or genes, excludes low-frequency or unnatural epitopes that are not relevant to circulating strains, and its intact protein antigens are more likely to be processed as in a natural infection.

The present invention results from the realization that a polyvalent set of antigens comprising synthetic viral proteins, the sequences of which provide maximum coverage of non-rare short stretches of circulating viral sequences, constitutes a good vaccine candidate. The invention provides a “genetic algorithm” strategy to create such sets of polyvalent antigens as mosaic blends of fragments of an arbitrary set of natural protein sequences provided as inputs. In the context of HIV, in certain embodiments the protein Gag is a candidate for such antigen. In certain embodiments the proteins Gag and Nef are candidates for such antigens. To expand coverage, in other embodiments, Pol and/or Env can also be used. The invention further provides optimized mosaic sets for these proteins.

The genetic algorithm strategy of the invention uses unaligned protein sequences from the general population as an input data set, and thus has the virtue of being “alignment independent”. It creates artificial mosaic proteins that resemble proteins found in nature—the success of the consensus antigens in small animals models suggest this works well. 9 Mers are the focus of the studies described herein, however, different length peptides can be selected depending on the intended target. In accordance with the present approach, 9 mers (for example) that do not exist in nature or that are very rare can be excluded—this is an improvement relative to consensus sequences since the latter can contain some 9 mers (for example) that have not been found in nature, and relative to natural strains that almost invariably contain some 9 mers (for example) that are unique to that strain. The definition of fitness used for the genetic algorithm is that the most “fit” polyvalent cocktail is the combination of mosaic strains that gives the best coverage (highest fraction of perfect matches) of all of the 9 mers in the population and is subject to the constraint that no 9 mer is absent or rare in the population.

The mosaics protein sets of the invention can be optimized with respect to different input data sets—this allows use of current data to assess virtues of a subtype or region specific vaccines from a T cell perspective.

The proteins/polypeptides/peptides and nucleic acids encoding these (“immunogens”) of the invention can be formulated into compositions with a pharmaceutically acceptable carrier and/or adjuvant using techniques well known in the art. Suitable routes of administration include systemic (e.g., intramuscular or subcutaneous), oral, intravaginal, intrarectal and intranasal. The immunogens of the invention can be chemically synthesized and purified using methods which are well known to the ordinarily skilled artisan. The immunogens can also be synthesized by well-known recombinant DNA techniques.

Nucleic acids encoding the immunogens of the invention can be used as components of, for example, a DNA vaccine wherein the encoding sequence is administered as naked DNA or, for example, a minigene encoding the immunogen can be present in a viral vector. The encoding sequences can be expressed, for example, in mycobacterium, in a recombinant chimeric adenovirus, or in a recombinant attenuated vesicular stomatitis virus. The encoding sequence can also be present, for example, in a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, another pox virus vector, recombinant polio and other enteric virus vector, Salmonella species bacterial vector, Shigella species bacterial vector, Venezuelean Equine Encephalitis Virus (VEE) vector, a Semliki Forest Virus vector, or a Tobacco Mosaic Virus vector. The encoding sequence, can also be expressed as a DNA plasmid with, for example, an active promoter such as a CMV promoter. Other live vectors can also be used to express the sequences of the invention. In certain embodiments, the nucleic acids of the invention are optimized for the expression vector or delivery mechanism. Expression of the immunogens of the invention can be induced in a patient's own cells, by introduction into those cells of nucleic acids that encode the immunogen, preferably using codons and promoters that optimize expression in human cells. Examples of methods of making and using DNA vaccines are disclosed in U.S. Pat. Nos. 5,580,859, 5,589,466, and 5,703,055. Examples of methods of codon optimization are described in Haas et al. Current Biology 6:315-324 (1996) and in Andre et al, J. Virol. 72(2):1497-1503 (1998).

It will be appreciated that adjuvants can be included in the compositions of the invention, or otherwise administered to enhance the immunogenic effect. Examples of suitable adjuvants include TLR agonists, such as but not limited to TLR-9 agonists, TLR-4 agonists, and TLR-7, 8 and 9 agonist combinations. In other embodiments the adjuvant is alum. Adjuvants can take the form of oil and water emulsions. Squalene adjuvants can also be used. Suitable adjuvants include, for example but not limited to, alum, poly IC, MF-59 or other squalene-based adjuvant, ASOIB, or other liposomal based adjuvant suitable for protein or nucleic acid immunization. In certain embodiments, TLR agonists are used as adjuvants. In some embodiments, the TLR agonist is a TLR4 agonist, such as but not limited to GLA/SE. In other embodiment, adjuvants which break immune tolerance are included in the immunogenic compositions. In some embodiments the adjuvant is TLR7 or a TLR7/8 agonist, or a TLR-9 agonist, or a combination thereof. See PCT/US2013/029164. The invention also contemplates any suitable combination of agonists.

The composition of the invention comprises an immunologically effective amount of the immunogen of this invention, or nucleic acid sequence encoding same, in a pharmaceutically acceptable delivery system. The compositions can be used for prevention and/or treatment of virus infection (e.g. HIV infection). As indicated above, the compositions of the invention can be formulated using adjuvants, emulsifiers, pharmaceutically-acceptable carriers or other ingredients routinely provided in vaccine compositions. Optimum formulations can be readily designed by one of ordinary skill in the art and can include formulations for immediate release and/or for sustained release, and for induction of systemic immunity and/or induction of localized mucosal immunity (e.g, the formulation can be designed for intranasal, intravaginal or intrarectal administration). As noted above, the present compositions can be administered by any convenient route including subcutaneous, intranasal, oral, intramuscular, or other parenteral or enteral route. The immunogens can be administered as a single dose or multiple doses. Optimum immunization schedules can be readily determined by the ordinarily skilled artisan and can vary with the patient, the composition and the effect sought.

The invention contemplates the direct use of both the immunogen of the invention and/or nucleic acids encoding same and/or the immunogen expressed as indicated above. For example, a minigene encoding the immunogen can be used as a prime and/or boost.

In certain aspects the invention includes any and all amino acid sequences disclosed herein, as well as nucleic acid sequences encoding same (and nucleic acids complementary to such encoding sequences). In other aspects, the invention is directed to methods of inducing immune response to HIV-1 comprising administering the amino acid and/or nucleic acid sequences as T-cell components of an immunogenic composition. In certain embodiments, the induced immune response reduces the risk of a viral infection. In other embodiments, the induced immune response treats a viral infection, for example but not limited to reducing viral load.

In certain aspects the invention provides M group mosaic pairs for every HIV of proteins based on the September 2013 Los Alamos database alignment. The bivalent mosaics described herein are designed for T-cell response. In certain embodiments these are included as T-cell components in immunogenic compositions and methods to induce immune response to HIV-1. HIV-1 specific T-cells are likely to be crucial to an HIV-1-specific vaccine response: CTL responses are correlated with slow disease progression in humans (Oxenius et al. J. Infect. Dis. 189: 1199-1208 (2004)), and the importance of CTL responses in non-human primate vaccination models is well-established. Vaccine elicited cellular immune responses help control pathogenic SIV or SHIV, and reduce the likelihood of disease after challenge with pathogenic virus (Barouch et al, Science 290:486-492 (2000)). Temporary depletion of CD8+ T cells results in increased viremia in SIV-infected rhesus macaques (Schmitz et al, Science 283:857-860 (1999)). Furthermore, the evolution of escape mutations has been associated with disease progression, indicating that CTL responses help constrain viral replication in vivo (Barouch et al, J. Virol. 77:7367-7375 (2003)), and so vaccine-stimulated memory responses that could block potential escape routes may be of value. While the highly variable Envelope (Env) is the primary target for neutralizing antibodies against HIV, and vaccine antigens will also need to be tailored to elicit these antibody responses (Moore & Burton. Nat. Med. 10:769-771 (2004)), T-cell vaccine components can target more conserved proteins to trigger responses that are more likely to cross-react. But even the most conserved HIV-1 proteins are diverse enough that variation will be an issue. Artificial central-sequence vaccine approaches, consensus and ancestral sequences (Gaschen et al, Science 296:2354-2360 (2002), Gao et al, J. Virol. 79:1154-1163 (2005), Doria-Rose et al, J. Virol, 79: 11214-11224 (2005)), which essentially “split the differences” between strains, show promise, stimulating responses with enhanced cross-reactivity compared to natural strain vaccines (Gao et al, J. Virol. 79:1154-1163 (2005)) (Liao et al. and Weaver et al., submitted.) Nevertheless, even central strains cover the spectrum of HIV diversity to a very limited extent, and consensus-based peptide reagents fail to detect many autologous CD8+ T-cell responses (Altfeld et al, J. Virol. 77:7330-7340 (2003)).

A single amino acid substitution can mediate T-cell escape, and as one or more amino acids in many T-cell epitopes differ between HIV-1 strains, the potential effectiveness of responses to any one vaccine antigen is limited. Whether a particular mutation will diminish T-cell cross-reactivity is epitope- and T-cell-specific, although some changes can broadly affect between-clade cross-reactivity (Norris et al, AIDS Res. Hum. Retroviruses 20:315-325 (2004)). Including more variants in a polyvalent vaccine could enable responses to a broader range of circulating variants. It could also prime the immune system against common escape variants (Jones et al, J. Exp. Med. 200:1243-1256 (2004)); escape from one T-cell receptor might create a variant that is susceptible to another (Lee et al, J. Exp. Med. 200:1455-1466 (2004)), thus stimulating polyclonal responses to epitope variants may be beneficial (Killian et al. AIDS 19:887-896 (2005)). Immune escape involving avenues that inhibit processing (Milicic et al, J. Immunol. 175:4618-4626 (2005)) or HLA binding (Ammaranond et al, AIDS Res. Hum. Retroviruses 21:395-397 (2005)) prevent epitope presentation, and in such cases the escape variant could not be countered by a T-cell with a different specificity. However, it is possible the presence of T-cells that recognize overlapping epitopes may in some cases block these even escape routes.

In certain aspects, the invention provides bivalent mosaic HIV polypeptides and nucleic acids sequences encoding these, as components of a vaccine, wherein the mosaics are designed to provide optimal coverage for CD4 and CD8 responses to HIV in order to promote the elimination of virus-infected T cells and macrophages at the time of transmission, and to promote the induction of optimal T cell help for antibody induction by a B cell immunogen vaccine.

The bivalent mosaic HIV genes could be administered as a DNA prime, modified vaccinia Ankara (MVA) regimen, or as multiple DNA or mRNA immunizations. In certain embodiments the nucleic acid sequences are codon optimized for optimal expression in a host cell, for example a mammalian cell, a rBCG cell, viral vector, or any other suitable expression system.

Nucleotide-based vaccines offer a flexible vector format to immunize against virtually any protein antigen. Currently, two types of genetic vaccination are available for testing-DNAs and mRNAs.

In certain aspects the invention contemplates using immunogenic compositions wherein immunogens are delivered as DNA. See Graham B S, Enama M E, Nason M C, Gordon U, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device.

In certain embodiments, the DNA is inserted in vectors. The DNA is delivered using a suitable vector for expression in mammalian cells. In certain embodiments the nucleic acids encoding these mosaics are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (Barouch D H, et al. Nature Med. 16: 319-23, 2010), recombinant mycobacteria (i.e., rBCG or M smegmatis) (Yu, J S et al. Clinical Vaccine Immunol. 14: 886-093, 2007; ibid 13: 1204-11, 2006), and recombinant vaccinia type of vectors (Santra S. Nature Med. 16: 324-8, 2010), for example but not limited to ALVAC, replicating (Kibler K V et al., PLoS One 6: e25674, 2011 Nov. 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54j 115-121; Arnaoty et al., Chapter 17 in Yves Bigot (ed.), Mobile Genetic Elements: Protocols and Genomic Applications, Methods in Molecular Biology, vol. 859, pp293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See e.g. InCellArt research and development materials.

Described herein are nucleic and amino acids sequences of HIV-1 envelopes. In certain embodiments, the described HIV-1 envelope sequences are gp160s. In certain embodiments, the described HIV-1 envelope sequences are gp120s. Other sequences, for example but not limited to gp145s, gp140s, both cleaved and uncleaved, gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . ”. In case of CH505 T/F Env as an example, 8 amino acids (italicized and underlined in the below sequence) were deleted:

MRVMGIQRNYPQWWIWSMLGFWMLMICNGMWVTVYYGVPVWKEAKTTLFC
ASDAKAYEKEVHNVWATHACVPTDPNPQE...
(rest of envelope sequence is indicated as “...”).

In other embodiments, the delta N-design described for CH505 T/F envelope can be used to make delta N-designs of other CH505 envelopes. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 11, amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain embodiments, the invention provides envelope sequences, amino acid sequences and the corresponding nucleic acids, and in which the V3 loop is substituted with the following V3 loop sequence TRPNNNTRKSIRIGPGQTFY ATGDIIGNIRQAH. This substitution of the V3 loop reduced product cleavage and improves protein yield during recombinant protein production in CHO cells.

In certain aspects the invention contemplates using immunogenic compositions wherein immunogens are delivered as recombinant proteins. Various methods for production and purification of recombinant proteins suitable for use in immunization are known in the art.

The immunogenic compositions can also be administered as a protein boost in combination with a variety of nucleic acid primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramascular (IM), via subcutaneous, via intravenous, via nasal, via mucosal routes.

The compositions can be formulated with appropriate carriers and adjuvants using techniques to yield compositions suitable for immunization. The compositions can include an adjuvant, such as, for example but not limited to, alum, poly IC, MF-59 or other squalene-based adjuvant, ASOIB, or other liposomal based adjuvant suitable for protein or nucleic acid immunization. In certain embodiments, TLR agonists are used as adjuvants. In other embodiment, adjuvants which break immune tolerance are included in the immunogenic compositions.

Any of the genes in the bivalent mosaic genome set, or combinations thereof, can be used in methods to induce immune response to HIV-1. In certain embodiments, the invention contemplates combinations of envelope and Gag and Vif mosaic genes.

In certain embodiments, an optimal HIV vaccine will not only include a bivalent mosaic T cell component for optimal T cell help for antibody induction but would also include a vaccine immunogens for inducing broadly neutralizing antibodies such as the CH505 swarm of Envs (U.S. Application Ser. No. 61/955,402, the contents of which is herein incorporated by reference in its entirety, e.g. all sequences disclosed therein, all envelope selections in the Examples) for induction of CD4 binding site broad neutralizing antibodies. Other swarm vaccines that could be co-administered with the mosaic T cell immunogens of the invention could swarm of Envs for induction of V3 glycan mabs (e.g. sequences from CH694, U.S. Patent Application 61/883,561, ex. FIG. 10, the entire contents of which is herein incorporated by reference in its entirety), the CH848 envelopes for inducing the V3 glycan mAbs, and the swarm of Envs for inducing CD4 binding site mAbs (e.g. envelope sequences selected from CH1754 envelopes as disclosed in U.S. Ser. Appl. No. 61/884,014 the contents of which is herein incorporated by reference in its entirety,).

For example, the combined T and B cell vaccines could be administered as a genetic prime with the mosaic bivalent vaccine genes expressed as DNAs or mRNAs co-administered with CH505 Envs as either proteins, DNAs, mRNAs or expressed in pox virus vectors such as modified vaccinia Ankara or NYVAC.

For induction of multiple species of antibodies, the CH505 envs could be combined with the CH848 Envs to have a B cell immunogen capable of inducing both CD4 binding site and V3 glycan site antibodies. the CH505 Envs also have N156 and N160 glycans and bind the V1V2 bnAb PG9. However, the PG9 unmutated common ancestor doesn't bind the CH505 Envs, but the synthetic V2 peptide glycan does bind the PG9 UCA (Alam, PNAS 110: 18214-9, 2013). Thus to simultaneously stimulate CD4 binding site antibodies (with CH505 Envs), V3 glycan antibodies (with CH848 sequential Envs), and V1V2 glycan antibodies (with priming with V2 peptide glycans and boosting with CH505 Envs) one can envision a multicomponent immunogen capable of inducing three specificities of bnAbs. Inducing at least more than one specificity of bnAb will be critical to prevent escape of the T/F virus.

The invention is further described in the following non-limiting examples.

### EXAMPLES

### Example 1

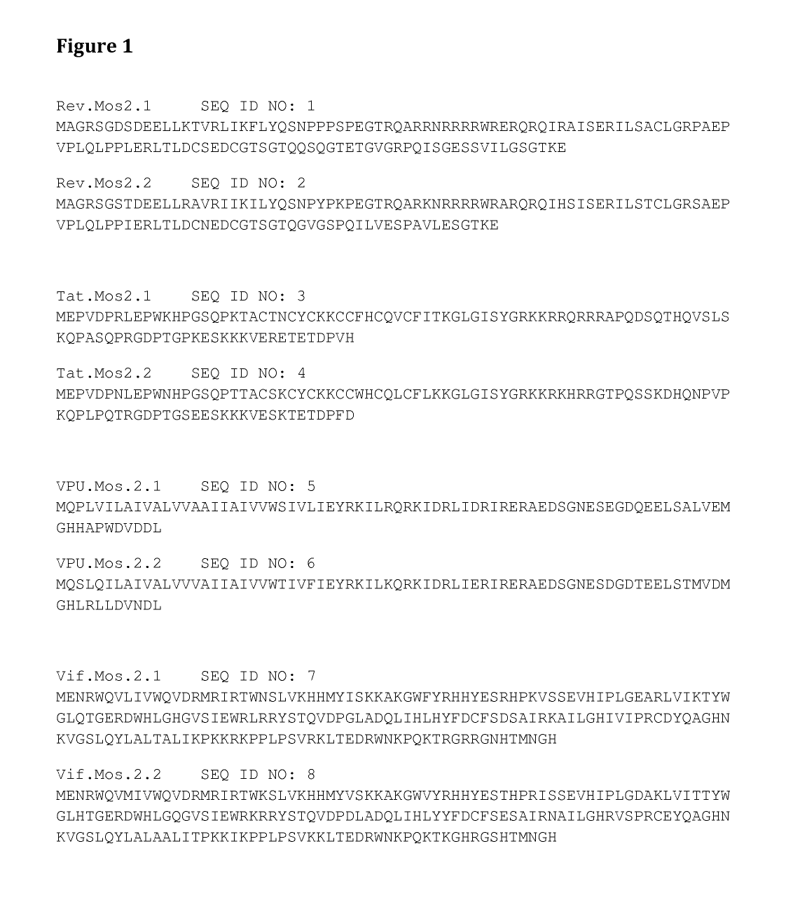

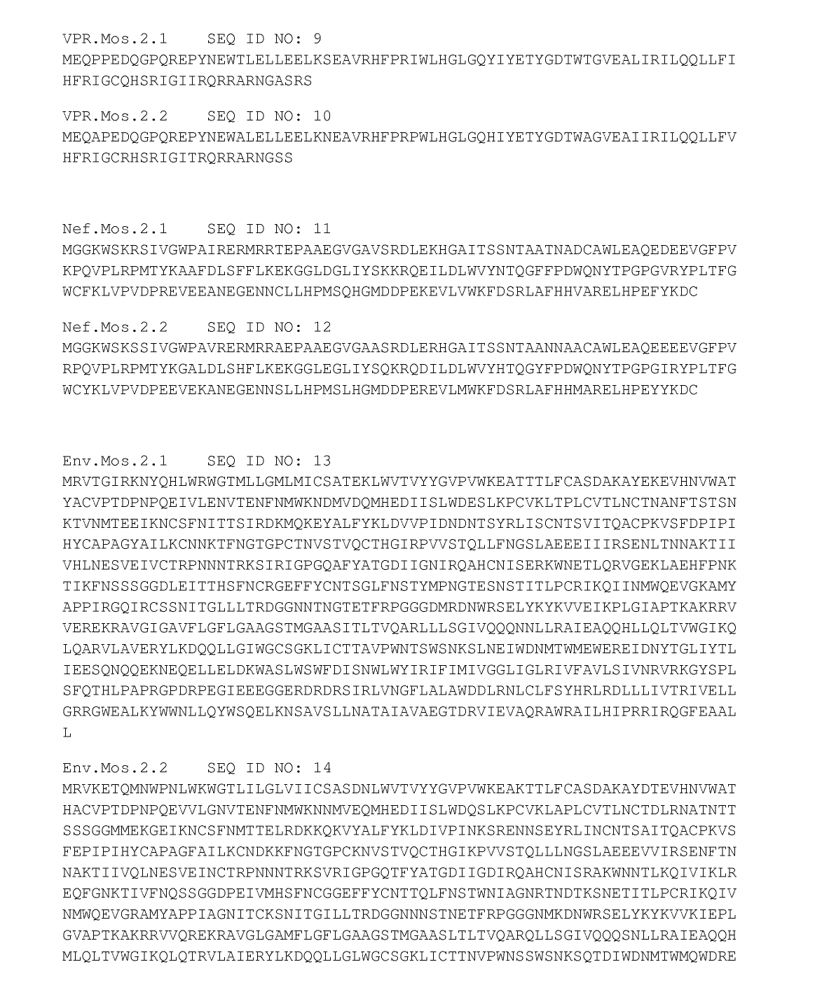

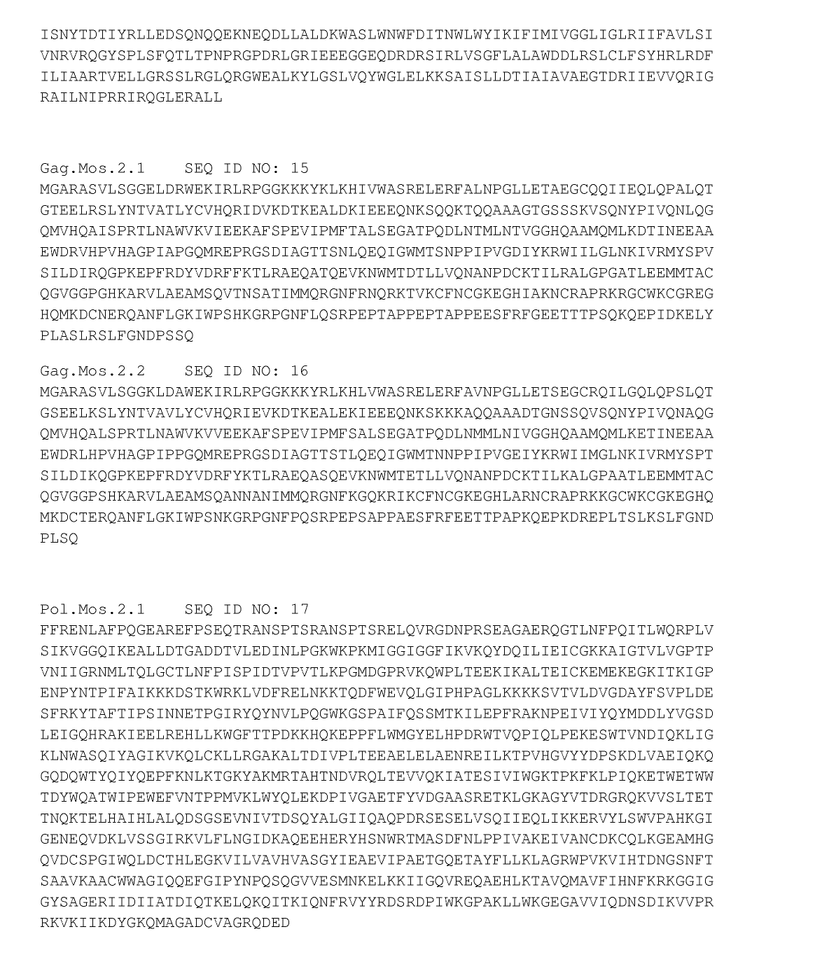

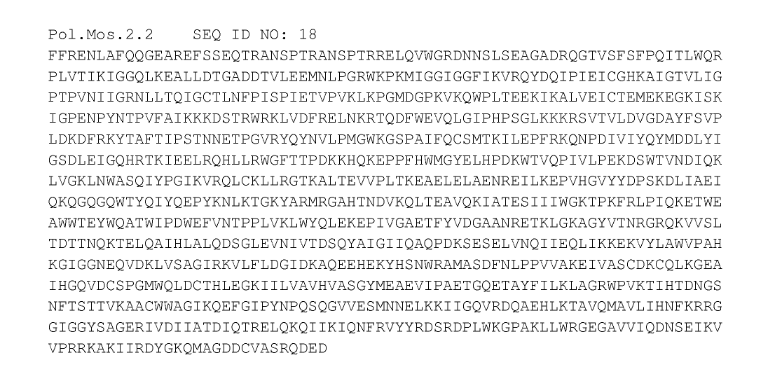

Using previously described genetic algorithm (U.S. Pat. No. 7,951,377, Ex. 1, the contents of which application is herein incorporated by reference in its entirety,) and the September 2013 HIV sequence database (URL: hiv-dot-lanl-dot-gov), a set of bivalent optimized sequences for every HIV protein was designed (FIG. 1). The code used for performing these analyses are available at: ftp://ftp-t10/pub/btlk/mosaics. In certain embodiments these sequences could be modified to make them suitable for use in immunogenic composition in various vaccination schedules. In certain embodiments modification include eliminating cleavage/fusion activity in Env, eliminating catalytic activity in Pol, eliminating myristylation sites in Nef. In other embodiments, the invention provides constructing fusion constructs including but not limited to GagNef, GagPol, or GagPolNef; GagVif, or GagVifEnv, or any other combination of the HIV genes.

### Example 2

The bivalent M group mosaic designs of the invention, for example mosaic envelope, can be compared to M consensus sequences and/or optimal natural clade C (or clade B) sequences. The M consensus envelope sequence (CON-S) represent synthetic sequence that represent the consensus of circulating viruses worldwide. The 2-valent M mosaic sequences are described in FIG. 1. An optimal natural clade C sequences are naturally occurring sequences from actual clade C HIV-1 viruses that are the most “consensus-like” in character. Cellular immune breadth can be assayed by any suitable assay. In one embodiment, it can be assessed by evaluating the number of responding peptides from the global potential T cell epitope (PTE) peptide set. The PTE peptides represent >85% of global HIV-1 sequences and are freely available from the NIH.

In one embodiment, where the antigens are delivered as DNA, for example in a NYVAC vector, to an animal, for example a non-human primate, the bivalent mosaic designs of the invention will outperform the immunogenicity, as measured by breadth/number and depth/diversity of the PTE peptide, of the consensus and optimal natural sequences.

### Example 3

One of the major obstacles to developing an efficacious preventive HIV-1 vaccine is the challenge of inducing broadly neutralizing antibodies (bnAbs) against the virus. There are several reasons why eliciting bnAbs has been challenging and these include the conformational structure of the viral envelope, molecular mimicry of host antigens by conserved epitopes which may lead to the suppression of potentially useful antibody responses, and the high level of somatic mutations in the variable domains and the requirement for complex maturation pathways [1-3]. It has been shown that up to 25% of HIV-1-infected individuals develop bnAbs that are detected 2-4 years after infection. To date, all bnAbs have one or more of these unusual antibody traits: high levels of somatic mutation, autoreactivity with host antigens, and long heavy chain third complementarity determining regions (HCDR3s)—all traits that are controlled or modified by host immunoregulatory mechanisms. Thus, the hypothesis has been put forth that typical vaccinations of single primes and boosts will not suffice to be able to induce bnAbs; rather, it will take sequential immunizations with Env immunogens, perhaps over a prolonged period of time, to mimic bnAb induction in chronically infected individuals [4].

A process to circumvent host immunoregulatory mechanisms involved in control of bnAbs is termed B cell lineage immunogen design, wherein sequential Env immunogens are chosen that have high affinities for the B cell receptors of the unmutated common ancestor (UCA) or germline gene of the bnAb clonal lineage [4]. Envs for immunization can either be picked randomly for binding or selected, as described herein, from the evolutionary pathways of Envs that actually give rise to bnAbs in vivo. Liao and colleagues recently described the co-evolution of HIV-1 and a CD4 binding site bnAb from the time of seroconversion to the development of plasma bnAb induction, thereby presenting an opportunity to map out the pathways that lead to generation of this type of CD4 binding site bnAb [5]. They showed that the single transmitted/founder virus was able to bind to the bnAb UCA, and identified a series of evolved envelope proteins of the founder virus that were likely stimulators of the bnAb lineage. Thus, this work presents the HVTN with an opportunity to vaccinate with naturally-derived viral envelopes that could drive the desired B-cell responses and induce the development of broad and potent neutralizing antibodies. While the human antibody repertoire is diverse, it has been found that only a few types of B cell lineages can lead to bnAb development, and that these lineages are similar across a number of individuals [6,7]. Thus, it is feasible that use of Envs from one individual will generalize to others.

The approach in this concept sheet to address the challenge of eliciting broadly neutralizing antibodies in vaccinees involves selecting the Env immunogens, among multitude of diverse viruses that induced a CD4 binding site bnAb clonal lineage in an HIV-infected individual, by making sequential recombinant Envs from that individual and using these Envs for vaccination. The B-cell lineage vaccine strategy thus includes designing immunogens based on unmutated ancestors as well as intermediate ancestors of known bnAb lineages. A candidate vaccine could use transmitted/founder virus envelopes to, at first, stimulate the beginning stages of a bnAb lineage, and subsequently boost with evolved Env variants to recapitulate the high level of somatic mutation needed for affinity maturation and bnAb activity. The goal of such a strategy is to selectively drive desired bnAb pathways.

Liao et al demonstrated that in the CHAVI CH505 bnAb individual, the CH103 CD4 binding site bnAb lineage started with the lineage members first developing autologous neutralizing antibody activity, and then as the CH505 Env mutated, it developed bnAb activity. Thus, the first step of bnAb development is the development of the ability to neutralize the transmitted/founder virus.

The CH505 transmitted/founder (T/F) virus that we propose to use in Trial 1 in the concept has been tested in rhesus macaques; after 3 immunizations it induced plasma antibodies that neutralized the T/F virus and an early (week 4) T/F variant with only one mutation. In addition, flow phenotypic analysis of memory B cells in CH505 T/F Env-immunized rhesus macaques has demonstrated the presence of antigen-specific memory B cells that bind the Env protein RSC3 but not the RSC3 371 mutant protein [8], strongly indicating B cells that have begun a CD4 binding site bnAb lineage.

In certain embodiments, the CH505 virus used is w004.03 instead of CH505 T/F.

Broadly neutralizing antibodies likely will not be induced by a single Env, and even a mixture of polyvalent random Envs (e.g. HVTN 505) is unlikely to induce bnAbs. Rather, immunogens must be designed to trigger the UCAs of bnAb lineages to undergo initial bnAb lineage maturation, and then use sequential immunogens to fully expand the desired lineages. The proposed trial will represent the first of many experimental clinical trials testing this concept in order to develop the optimal set of immunogens to drive multiple specificities of bnAbs. The HVTN will be at the cutting edge of this effort.

The concept is applicable to driving CD4 binding site lineage in multiple individuals due to the convergence of a few bnAb motifs among individuals. The adjuvant will be the GSK AS01E adjuvant containing MPL and QS21. Other suitable adjuvants can be used. This adjuvant has been shown by GSK to be as potent as the similar adjuvant AS01B but to be less reactogenic using HBsAg as vaccine antigen [Leroux-Roels et al., IABS Conference, April 2013, 9].

Schema Trial (Sequential doses of HIV envelopes see e.g. U.S. Application Ser. No. 61/955,402, the contents of which is herein incorporated by reference in its entirety, e.g. all sequences disclosed therein, all envelope selections in the Examples and bivalent mosaics): NHP study; clinical trial with healthy human subjects

| Study | | Month 0 | Month 1 | Month 3 | Month 6 | Month 12 | Month 18 |
|---|---|---|---|---|---|---|---|
| arm | N | (Day 0) | (Day 28) | (Day 84) | (Day 168) | (Day 364) | (Day 546) |
| Group 1 | 12 | 100 mcg T/F | 100 mcg | 100 mcg | 100 mcg | 100 mcg | 100 mcg |
| | | | 53 | 78 | 100 | Swarm | Swarm |
| Group 2 | 12 | 100 mcg | 100 mcg | 100 mcg | 100 mcg | 100 mcg | 100 mcg |
| | | T/F + | 53 + | 78 + | 100 + | Swarm | Swarm |
| | | 4 mg DNA | 4 mg DNA | 4 mg DNA | 4 mg DNA | | |
| | | mosaic | mosaic | mosaic | mosaic | | |
| Group 3 | 12 | 100 mcg | 50 mcg T/F + | 33 mcg T/F + | 100 mcg | 100 mcg | 100 mcg |
| | | T/F | 50 mcg 53 | 33 mcg 53 + | Swarm | Swarm | Swarm |
| | | | | 33 mcg 78 | | | |
| Group 4 | 12 | 100 mcg | 100 mcg | 100 mcg | 100 mcg | 100 mcg | 100 mcg |
| | | Swarm | Swarm | Swarm | Swarm | Swarm | Swarm |
| Group 5 | 12 | placebo | placebo | placebo | placebo | placebo | placebo |
| Total | 60 | | | | | | |
| | (48/12) | | | | | | |

Schema Trial (Sequential doses of HIV envelopes and bivalent mosaics): NHP study; clinical trial with healthy human subjects

Notes: T/F=transmitted/founder protein; Swarm=mixture of T/F, 53, 78, and 100; Example protein doses included, total actual dose to be informed by dose finding studies.

Products: CH505TF: transmitted/founder HIV gp120 with AS01E; CH505w53.1: week 53 HIV gp120 with AS01E; CH505w78.33: week 78 HIV gp120 with AS01E; CH505w100.6: week 100 HIV gp120 with AS01E

DNA Mosaic: bivalent vaccine composed of mosaic Env. Mos.2.1 and Env.Mos.2.1 that optimizes for global coverage. See FIG. 1. All express gp160 Env protein. Any other suitable mosaic as described herein could be used in the trial.

Placebo: sodium chloride for injection.

Statistical Considerations

Accrual and sample size calculations: Recruitment into trial #2 will target 60 healthy, HIV-uninfected adults aged 18 to 50 years old at low risk of HIV infection in regions where clade B is the predominant clade. Enrollment will be concurrent with receiving the first study vaccination, thus all participants will provide some safety data. For immunogenicity analyses, however, it is possible that data may be missing for various reasons such as participants terminating from the study early, problems in shipping specimens, or low cell viability of processed peripheral blood mononuclear cells (PBMCs). Immunogenicity data from 11 phase 1 and 1 phase 2a HVTN trials, which began enrolling after June 2005 (data as of June 2011), indicate that 10% is a reasonable estimate for the rate of missing data. For this reason, the sample size calculations below account for 10% of enrolled participants having missing data for the primary immunogenicity endpoint.

Sample size calculations for safety: The ability of the study to identify SAEs can be expressed by the true event rate above which at least 1 event would likely be observed and the true event rate below which no events would likely be observed. Specifically, in each vaccine arm of the study (n=12), there is a 90% chance of observing at least 1 event if the true rate of such an event is 17.5% or more; and there is a 90% chance of observing no events if the true rate is 0.8% or less. In all vaccine arms of the study combined (n=36), there is a 90% chance of observing at least 1 event if the true rate of such an event is 6.2% or more; and there is a 90% chance of observing no events if the true rate is 0.2% or less.

Sample size calculations for immunogenicity: To address antibody endpoints, the analysis will descriptively summarize binding response positivity call rates and test superiority of the magnitude and breadth of the IgG binding Ab response to a panel of gp120 proteins for each of comparison, using a two-sided Wilcoxon rank sum test with 2.5% type-I error rate per comparison. The sample size of 12 vaccinees per group will give 80% power to detect a true difference of 1.82 standard deviations (SDs) between the mean non-zero responses and 90% power to detect a true difference of 2.04 SDs. These calculations assume a 10% loss-to-follow-up rate and the (94%) response rate observed in the HVTN 088 vaccine recipients. The same approach will be used to test superiority of the magnitude of the IgG binding Ab response to each individual gp120 antigen in the panel.

### REFERENCES FOR EXAMPLE 3

1. Mascola J R, Haynes B F. HIV-1 neutralizing antibodies: understanding nature's pathways. Immunol Rev 2013; 254:225-44.
2. Verkoczy L, Kelsoe G, Moody M A, Haynes B F. Role of immune mechanisms in induction of HIV-1 broadly neutralizing antibodies. Curr Opin Immunol 2011; 23:383-90.
3. Verkoczy L, Chen Y, Zhang J, Bouton-Verville H, Newman A, Lockwood B, Scearce R M, Montefiori D C, Dennison S M, Xia S M, Hwang K K, Liao H X, Alam S M, Haynes B F. Induction of HIV-1 broad neutralizing antibodies in 2F5 knock-in mice: selection against membrane proximal external region-associated autoreactivity limits T-dependent responses. J Immunol 2013; 191:2538-50.
4. Haynes B F, Kelsoe G, Harrison S C, Kepler T B. B-cell-lineage immunogen design in vaccine development with HIV-1 as a case study. Nat Biotechnol 2012; 30:423-33.
5. Liao H X, Lynch R, Zhou T, Gao F, Alam S M, Boyd S D, Fire A Z, Roskin K M, Schramm C A, Zhang Z, Zhu J, Shapiro L, Mullikin J C, Gnanakaran S, Hraber P, Wiehe K, Kelsoe G, Yang G, Xia S M, Montefiori D C, Parks R, Lloyd K E, Scearce R M, Soderberg K A, Cohen M, Kamanga G, Louder M K, Tran L M, Chen Y, Cai F, Chen S, Moquin S, Du X, Joyce M G, Srivatsan S, Zhang B, Zheng A, Shaw G M, Hahn B H, Kepler T B, Korber B T, Kwong P D, Mascola J R, Haynes B F. Co-evolution of a broadly neutralizing HIV-1 antibody and founder virus. Nature 2013; 496:469-76.
6. Morris L, Chen X, Alam M, Tomaras G, Zhang R, Marshall D J, Chen B, Parks R, Foulger A, Jaeger F, Donathan M, Bilska M, Gray E S, Abdool Karim S S, Kepler T B, Whitesides J, Montefiori D, Moody M A, Liao H X, Haynes B F. Isolation of a human anti-HIV gp41 membrane proximal region neutralizing antibody by antigen-specific single B cell sorting. PLoS One 2011; 6:e23532.
7. Zhou T, Zhu J, Wu X, Moquin S, Zhang B, Acharya P, Georgiev I S, Altae-Tran H R, Chuang G Y, Joyce M G, Do K Y, Longo N S, Louder M K, Luongo T, McKee K, Schramm C A, Skinner J, Yang Y, Yang Z, Zhang Z, Zheng A, Bonsignori M, Haynes B F, Scheid J F, Nussenzweig M C, Simek M, Burton D R, Koff W C, Mullikin J C, Connors M, Shapiro L, Nabel G J, Mascola J R, Kwong P D. Multidonor analysis reveals structural elements, genetic determinants, and maturation pathway for HIV-1 neutralization by VRC01-class antibodies. Immunity 2013; 39:245-58.
8. Lynch R M, Tran L, Louder M K, Schmidt S D, Cohen M, Dersimonian R, Euler Z, Gray E S, Abdool K S, Kirchherr J, Montefiori D C, Sibeko S, Soderberg K, Tomaras G, Yang Z Y, Nabel G J, Schuitemaker H, Morris L, Haynes B F, Mascola J R. The Development of CD4 Binding Site Antibodies During HIV-1 Infection. J Virol 2012; 86:7588-95.
9. Leroux-Roels I, Koutsoukos M, Clement F, Steyaert S, Janssens M, Bourguignon P, Cohen K, Altfeld M, Vandepapeliere P, Pedneault L, McNally L, Leroux-Roels G, Voss G. Strong and persistent CD4+ T-cell response in healthy adults immunized with a candidate HIV-1 vaccine containing gp120, Nef and Tat antigens formulated in three Adjuvant Systems. Vaccine 2010; 28:7016-24.

What is claimed is:

1. A composition comprising a nucleic acid encoding any one of the mosaic polypeptides of FIG. 1, or a combination thereof.

2. A composition comprising a bivalent set of nucleic acids encoding two mosaic polypeptides of FIG. 1, or any combination thereof, wherein the set of two mosaic polypeptides correspond to the same viral gene product.

3. The composition of claim 1 comprising nucleic acids encoding Gag.Mos.2.1, Gag.Mos.2.2, or the combination thereof.

4. The composition of claim 1 comprising nucleic acids encoding Env.Mos.2.1, Env.Mos.2.2, or the combination thereof.

5. The composition of claim 1 comprising nucleic acids encoding Vif.Mos.2.1, Vif.Mos.2.2, or the combination thereof.

6. The composition of claim 1 comprising nucleic acids encoding Pol.Mos.2.1, Pol.Mos.2.2, or the combination thereof.

7. The composition of claim 1 comprising nucleic acids encoding Nef.Mos.2.1, Nef.Mos.2.2, or the combination thereof.

8. The composition of claim 1 comprising nucleic acids encoding Gag.Mos.2.1, Gag.Mos.2.2, Vif.Mos.2.1, Vif.Mos.2.2, or the combination thereof.

9. The composition of claim 1, further comprising Nef.Mos.2.1, Nef.Mos.2.2, or the combination thereof.

10. The composition of claim 1, further comprising Env.Mos.2.1, Env.Mos.2.2, or the combination thereof.

11. The composition of claim 1 to 10, further comprising an adjuvant.

12. The composition of claim 1 to 10, wherein the composition further comprises an HIV-1 immunogen suitable to elicit a humoral response.

13. A method of inducing an immune response in a subject comprising administering to the subject an amount of the composition of any one of claim 1-12 in an amount sufficient to effect such induction.

14. The method of claim 12, wherein the composition further comprises an immunogen, for example but not limited to HIV1 envelope, suitable to elicit a humoral response.
